(19) United States
(12) Patent Application Publication
(10) Pub. No.: US 2026/0315636 A1
(43) Pub. Date: Oct. 15, 2026

(54) CARTRIDGE DEVICE AND SYSTEM FOR USE IN AN APPARATUS FOR AUTOMATED STERILITY TESTING OF RADIOPHARMACEUTICALS BY MEANS OF MEMBRANE FILTRATION, AS WELL AS ARRANGEMENT AND USE OF THE DEVICE AND OF THE SYSTEM IN SUCH AN APPARATUS

(71) Applicant: CUP LABORATORIEN DR. FREITAG GMBH, Radeberg (DE)

(72) Inventors: Dirk FREITAG-STECHL, Dresden (DE); Kerstin TAUTZ, Oranienburg (DE)

(21) Appl. No.: 19/119,101

(22) PCT Filed: Oct. 10, 2023
(86) PCT No.: PCT/EP2023/077975

§ 371 Date: Apr. 8, 2025

(30) Foreign Application Priority Data
Oct. 12, 2022 (DE) 10 2022 126 549.8

(51) Int. Cl. A61L 2/28; A61L 103/10; B01D 63/00; C12Q 1/22

(52) U.S. Cl. CPC A61L 2/28; B01D 63/00; C12Q 1/22; A61L 2103/10; B01D 2313/08; B01D 2313/105; B01D 2313/125; B01D 2313/13; B01D 2313/18; B01D 2313/243; B01D 2313/44; B01D 2313/58; B01D 2317/04

(57) ABSTRACT

The present invention relates to a cartridge device (1) for use in an apparatus (100) for the automated sterility testing of radiopharmaceuticals by means of membrane filtration, a system and an arrangement comprising the cartridge device (1) according to the invention, and the use of the cartridge device according to the invention or of the system according to the invention for the automated sterility testing of radiopharmaceuticals by means of membrane filtration, in particular in a corresponding apparatus (100). The cartridge device (1) according to the invention comprises a distributor unit (2) with a base plate (12), valves (3a-3f), in particular three-way valves, lines (5ab-5ef) and connections (4a-4j), wherein the connections (4a-4j) can selectively communicate with one another via the valves (3a-3f) and lines (5ab-5ef), a first filter unit (7d) and at least five hoses (5a, 5b, 5d, 5f, 5g) that communicate with corresponding connections (4a, 4b, 4d, 4f, 4g) of the distributor unit (2), wherein one hose (5d) communicates with the first filter unit (7d) and four hoses (5a, 5b, 5f, 5g) are configured such that one hose thereof communicates in each case with a first external container with buffer solution (16a), an external container with nutrient solution (16b), an external radiation-shielded container for waste solution (16f) and an external radiation-shielded container for sample material (16g).

### TECHNICAL FIELD

The present invention relates to a cartridge device for use in an apparatus for the automated sterility testing of radiopharmaceuticals by means of membrane filtration, a system and an arrangement comprising the cartridge device according to the invention, and the use of the cartridge device according to the invention or of the system according to the invention for the automated sterility testing of radiopharmaceuticals by means of membrane filtration, in particular in a corresponding apparatus.

### BACKGROUND AND PRIOR ART

Sterility testing as according to the European Pharmacopoeia 11.0, 2.6.1 “Sterility” (Ph. Eur. 2.6.1—Sterility Testing) or the U.S. Pharmacopeia (USP <71>) forms an important part of the quality control of medicaments and medical products manufactured under aseptic conditions for use in humans or animals. Medicaments and medical products include aqueous solutions, soluble powders, oils and oily solutions, ointments and creams, infusion bags, implants, for example heart valves or stents, and surgical suture material. Compliance with aseptic test conditions (cleanroom grade A, see EU GMP Guidelines, Annex 1) is important for the sterility testing of medicaments and medical products and is often ensured by the use of an isolator (sterile box) to prevent contamination.

Sterility testing is preferably carried out using the membrane filtration method. The membrane filtration method is a method for the mechanical enrichment of microorganisms from any quantity of filterable test material. Even with minimal microbial content, this method enables precise microbial count determinations. In this method, a certain volume of liquid is filtered through a membrane filter with a defined pore size and the microbes contained therein are retained on the filter surface. The filter is then brought to a culture medium. The nutrients of the culture medium enable growth of the individual microbes into diagnosable colonies. Alternatively, the contaminated filters can be transferred into liquid culture media. Depending on the medium, an increase in microorganisms is indicated either by turbidity or, if indicator dyes are contained in the medium, by colour changes.

In the case of non-filterable solutions, the medicament or medical product is dissolved or diluted in aqueous or oily solvents and then filtered. The used solvent must not have a negative effect on the sterility testing, and in particular must not have any antimicrobial properties. In the case of non-soluble products (for example implants, surgical suture material), testing is carried out by means of the direct inoculation method, wherein the product is introduced directly into the culture medium. The sterility testing of radioactive medicaments has posed a problem to date.

In order to reduce the risk of contamination, many quality aspects can already be implemented during manufacture of the radiopharmaceuticals (grade C, grade A, automated processes, sterile single-use consumables, precursors with a low microbial load, periodic media fill procedures), but even these measures do not provide exemption from performing the legally required sterility testing of the end products in order to rule out risks for patients.

In the field of radiopharmacy, sterility testing must be carried out retrospectively due to the short half-life of the radionuclides and the incubation period of at least 10 days. The European Pharmacopoeia monographs (for example 68Ga-Edotreotide or 18F-PSMA) therefore accept post-release sterility testing, although studies presented in the past point out that testing (for instance membrane filtration) should be started as early as possible in order to reduce the likelihood of false negative results. The EANM Radiopharmacy Committee recommended the following back in 2010:“Sterility testing should start as soon as feasible after the completion of SSRP production. If the sample for sterility testing is held for a longer storage time, small-scale radiopharmacies should demonstrate that the longer period does not adversely affect the test results”. The U.S. Pharmacopoeia sets (with some exceptions) the start of the incubation period of the sample at not more than 30 hours after manufacture of a radiopharmaceutical.

Irrespective thereof, relevant guidelines (EU, US, AUS) recognise the need to test radiopharmaceuticals as soon as possible after manufacture. If literature, guidelines and manuals are compared, it seems difficult to justify the common practice of sending samples to an external company for sterility testing once decay has reached permissible low levels. In the case of 177Lu or 225Ac radiopharmaceuticals with a half-life of 6.7 and 10 days, respectively, this means that there are several months between manufacture and the start of testing, which is not in line with the requirement to start testing as quickly as possible.

Due to the challenges of handling and storing the radioactive filtrate, the performance of the membrane filtration method for radiopharmaceuticals has been problematic to date. The radioactive filtrates often remain in the isolators and take up valuable space until they decay to below the allowed limit, posing a potential contamination risk and a risk to operators. It is furthermore difficult to install conventional extraction systems for membrane filtration in the isolators since these are usually used for manufacturing radiopharmaceuticals. The sterility testing of radioactive substances, in particular radioactive pharmaceuticals and/or diagnostics, is therefore often carried out by means of the direct inoculation method. The disadvantage hereof is that testing is limited to a small sample quantity since the radioactivity is problematic for microbial testing. The sterility testing of radioactive pharmaceuticals by means of the direct inoculation method furthermore does not comply with the legal requirements according to the European Pharmacopoeia (Ph. Eur. 2.6.1—Sterility Testing), the U.S. Pharmacopeia (USP <71>) and Ph. Eur. monograph 04/2022:0125; a clear preference for membrane filtration is apparent therein.

Alternatively, the sterility testing of radioactive substances can be carried out after the radioactivity has decayed, in particular after approximately three months, using the membrane filter method. However, this method can only be used for radioactive substances with short-lived nuclides. Long-lived nuclides are excluded due to the high amount of time required. Current research results (see poster contribution: “Time-dependent sterility testing to determine the viability of microorganisms and autosterilisation in radiopharmaceutical therapeutics”, A. Gummesson et al., EANM'21 Virtual Congress, 20 to 23 October 2021) suggest that there is furthermore no guarantee that any microbiological impurities present in the medicament present in the preparation will survive the long period of time until start. A further disadvantage is that sterility testing several months after manufacture of the radioactive substances is not very informative, in particular due to the negative influence of radioactivity on microbial growth.

EP 3944867 A1 further discloses a system and a method for testing the sterility of radioactive materials, comprising an isolator and a radiation-shielded container for radioactive materials. When reference is made to an “isolator” or a “radiation-shielded container” in the present application, what is meant is in particular the isolator and radiation-shielded container, in particular waste container, known from EP 3944867 A1. The corresponding passages of disclosure in EP 3944867 A1 are therefore to be included in the disclosure of the present application.

The object of the present invention is, inter alia, to provide a device for the automated sterility testing of radiopharmaceuticals by means of membrane filtration, which is compatible with existing apparatus for manufacturing radiopharmaceuticals, which ensures sufficient radiation protection and which enables sterility testing to be carried out immediately after manufacture of the radiopharmaceuticals and in accordance with the currently applicable legal regulations in order to improve the quality and informative value of the test results and thus ultimately patient protection. The device should also be as cost-effective and easy to use as possible.

### DESCRIPTION OF THE INVENTION

The present invention is defined in the enclosed claims which disclose a cartridge device according to the invention, preferred embodiments thereof, a system according to the invention, an arrangement according to the invention and uses according to the invention.

The present invention is technologically closely associated with the sterility testing by means of membrane filtration as standardised in the European Pharmacopoeia 11.0, 2.6.1 “Sterility”, and provides, but is not limited to, a particularly suitable and advantageous cartridge device, optionally as a consumable material, for the safe and efficient performance thereof, in particular on radiopharmaceuticals.

Some of the terms used in the following are to be understood in particular as follows, but not limited thereto:

“Radiopharmaceuticals” are to be understood as medicaments that are used for diagnostic or therapeutic purposes and contain radioactive components so that they emit radioactive radiation. This term also includes “radiotherapeutics”.

In the present application, a “cartridge device” is to be understood as a device that is intended to be connected to an apparatus in order to fulfil a specific function. By using different cartridges, different functions can therefore be performed with the same apparatus. As already mentioned above, the cartridge device according to the invention is preferably configured and adapted for use with and in systems and devices for the sterility testing of radiopharmaceuticals by means of membrane filtration in accordance with the European Pharmacopoeia 11.0, 2.6.1 “Sterility”. However, other uses are not ruled out.

A “system” or “arrangement” is to be understood as the combination of a device with further elements that are useful for fulfilling the intended function. In the present application, these “functions” preferably relate to sterility testing by means of membrane filtration.

In the present application, an “apparatus” is to be understood as a machine that can be connected to various cartridges or other components in order to fulfil certain functions. The apparatus may consist of a plurality of modules that fulfil different functions. Using actuators such as actuating members, the apparatus can interact in an automated manner with corresponding components of the cartridge and actuate them.

A “radiation-shielded container” is to be understood as a container that can accommodate radioactive substances and largely prevents radiation from escaping through the container to the outside. It is in particular to be understood as a radiation-shielded container according to EP 3944867 A1. An “isolator”, also known as a “sterile box” or “glove box”, is to be understood as a system that is sealed hermetically and in a gas-tight manner from the surrounding workspace. A defined atmosphere for processing radioactive substances can be generated inside the isolator. It is in particular to be understood as an isolator according to EP 3944867 A1.

“Sample material” is preferably to be understood as a radiopharmaceutical on which sterility testing must be performed. It may, for example, be in the form of a solid, a powder or the like. “Buffer solution” is to be understood as a liquid that is mixed with the sample material or in which the sample material is dissolved in order to be able to carry out subsequent membrane filtration. “Nutrient solution” is to be understood as a liquid applied to the membrane filter after filtration of the buffer solution with the sample material dissolved therein in order to promote microbial growth. “Waste solution” is to be understood as the liquid that remains after filtration and must be disposed of.

“Communication” is to be understood as the establishment or existence of a fluid connection so that liquids or gases can be exchanged. In the present application, “communication” is preferably to be understood as “functionally connected” and includes, on the one hand, a physical or mechanical connection (e.g. attaching or pugging-in) of system components, which is also functional and allows the one-way or multi-way exchange of gases and liquids in particular. Where mentioned below, the corresponding device and system components are correspondingly arranged and configured therefor.

The cartridge device according to the invention is intended to be used in an apparatus or system for the automated sterility testing of radiopharmaceuticals by means of membrane filtration, optionally the sterility testing by means of membrane filtration as standardised in the European Pharmacopoeia 11.0, 2.6.1 “Sterility”. It comprises a distributor unit with a base plate, valves, in particular three-way valves, lines and connections, a first filter unit and at least five hoses. The lines optionally and preferably connect the various connections with and via the various valves, i.e. they are the means of connection between these components. The connections can selectively communicate with one another via the valves, while the hoses communicate with the corresponding connections of the distributor unit. In the present application, “selectively” is preferably to be understood as switchable between one another and flexibly selectable. Depending on the requirements during use, the various connections can therefore be connected or brought into communication with one another in a targeted and needs-based manner. “Corresponding” is preferably to be understood as a functional affiliation, for example a connection is assigned its own (“corresponding”) hose and a valve is assigned its own connection and vice versa, resulting in functional component pairings that communicate or are able to communicate with one another. Preferred component pairings and assignments are also shown by way of an example in the figures and are apparent therefrom. One hose furthermore communicates with the first filter unit and four hoses are furthermore configured such that one hose thereof can in each case communicate with a first external container for/with buffer solution, an external container for/with nutrient solution, an external radiation-shielded container for/with waste solution and an external radiation-shielded container for/with sample material.

The cartridge device according to the invention and in particular the corresponding components thereof are intended to be connected to or communicate with external components of an overall test system, for example the various aforementioned containers. Such an actual connection is realised as part of the “system for automated sterility testing” that is also in accordance with the invention. In this system, the various components of the cartridge device will be and are then actually functionally connected to or communicate with the corresponding system components (containers, etc.). The term “external” preferably refers to components that are not part of the cartridge device itself, but are components with which the cartridge device is functionally connected during use and thus forms an overall system, inter alia for sterility testing by means of membrane filtration.

In order to manufacture pharmaceuticals, in particular radiopharmaceuticals, cartridge devices are already used in conjunction with corresponding apparatus (modules) to generate, isolate and/or mix the individual components of the pharmaceuticals in a sterile and automated manner. Since the cartridge device according to the invention can be used in conjunction with the same apparatuses, these can be used for both the manufacture and the subsequent sterility testing of the pharmaceuticals, thus saving time and costs, e.g. for transportation to an external laboratory. The quality of the test results and personal protection are also improved thanks to the closed system and automation since, in contrast to manual testing, any contamination of the sample material and differences in performance of the sterility test as well as any contamination or radiation exposure of persons can be minimised. Membrane filtration furthermore makes it possible to separate the microbes from the radioactive material, such that the microbial load of the pharmaceuticals can already be determined immediately after their production and not only once the radioactivity has decayed, which further improves the quality of the results. In order to further improve radiation protection, the sterility testing by means of membrane filtration can be carried out in an isolator.

The cartridge device preferably further comprises a second filter unit and a further hose communicating with a corresponding connection of the distributor unit and the second filter unit.

The provision of two filter units has the advantage that the microbes remaining in the respective filter units can each be exposed to different incubation conditions, such as different culture media, incubation temperatures or incubation times, which can further improve the quality of the test results, in particular as regards microbial contamination by different types of microbe.

The cartridge device preferably further comprises a syringe for a syringe pump and a further hose communicating with a corresponding connection of the distributor unit and the syringe. In the present application, a “syringe pump” is to be understood as an arrangement consisting of a syringe and an additional actuation unit or syringe pump module. The actuation unit herein may be a motor-driven clamping device into which the syringe is inserted and the syringe plunger may be moved back and forth by a motor.

The provision of a syringe for a syringe pump means that predetermined quantities of liquid can be drawn in and released with high precision. This ensures that the predetermined mixing ratios of the respective liquids are precisely adhered to, as a result of which constant test conditions and thus high quality test results can be achieved. The cartridge device can optionally also have a syringe pump.

The cartridge device preferably also comprises a further hose communicating with a corresponding connection of the distributor unit and configured to communicate or be able to communicate with a second external container containing nutrient solution.

The provision of two nutrient solutions, in particular two different and unmixed nutrient solutions, makes it possible to expose the microbes remaining in the filter unit or filter units to different nutrient solutions, which can further improve the quality of the test results, in particular as regards microbial contamination by different types of microbe. Furthermore, if two filter units are provided, the first filter unit may be supplied with the first nutrient solution and the second filter unit with the second nutrient solution, such that the respective filter units can be used, for example, to determine the microbial contamination by different types of microbe.

The cartridge device preferably further comprises at least four or at least/preferably five cannulas attached to the distal end of the corresponding hoses, i.e. preferably remote/facing away from the distributor unit, the cannulas being configured and provided to communicate or be able to communicate with the external container for/with buffer solution, the first external container for/with nutrient solution, the second external container for/with nutrient solution, the external radiation-shielded container for/with waste solution and the external radiation-shielded container for/with sample material.

The provision of cannulas at the distal end of the hoses makes it easier to connect the hoses to the respective containers and ensures that no liquid can accidentally escape to the outside, thus largely preventing the contamination of persons, in particular by radioactive material.

The distributor unit preferably comprises at least six valves, preferably all in the form of three-way valves, and at least eight connections, optionally ten connections. These valves and connections can optionally be “interconnected” or communicate with one another, as defined below.

Apparatuses for manufacturing pharmaceuticals are normally configured to accommodate cartridge devices with six valves and eight or ten connections, and thus the cartridge device according to the invention, which has six valves and eight or ten connections, can also be used therewith. The claimed number of valves and connections is furthermore particularly suitable for the automated sterility testing of radiopharmaceuticals by means of membrane filtration.

It is furthermore preferred that the distributor unit comprises a first, second, third, fourth and fifth line, a first three-way valve configured to establish selective communication between a connection for supplying buffer solution, a connection for connection to the syringe for a syringe pump and the first line; a second three-way valve configured to establish selective communication between a connection for supplying a first nutrient solution, the first line and the second line; a third three-way valve configured to establish selective communication between a connection for supplying a second nutrient solution, the second line and the third line; a fourth three-way valve configured to establish selective communication between a connection for connection to the first filter unit, the third line and the fourth line; a fifth three-way valve configured to establish selective communication between a connection for connection to the second filter unit, the fourth line and the fifth line; and a sixth three-way valve configured to establish selective communication between a connection for the removal of waste solution, a connection for supplying sample solution and the fifth line.

The claimed arrangement of the six three-way valves, five lines and the respective connections for supplying the buffer solution, the sample solution and the two nutrient solutions, for removing waste solution and for connection to the syringe and the two filter units is particularly suitable for use in an existing apparatus for manufacturing pharmaceuticals and for the automated sterility testing of radiopharmaceuticals by means of membrane filtration.

At least one of the first, second, fourth and fifth lines is preferably an integral part of the distributor unit or the base plate, and the third line is preferably an external hose or an integral part of the distributor unit or the base plate, and/or at least one of the first, second, third, fourth, fifth and sixth three-way valves is preferably an integral part of the distributor unit or the base plate. Integral embodiments can be produced, for example, by additive manufacturing or 3D printing.

Configuring the first, second, fourth and fifth lines as an integral part of the distributor unit has the advantage that external lines or connecting hoses therefor can be avoided, which improves the compactness and robustness of the cartridge device. Configuring the third line as an external hose is structurally simpler and therefore cheaper to manufacture.

The distributor unit preferably furthermore comprises at least one attachment, preferably at least two attachments, with at least one of the lines being arranged inside the attachment or one of the attachments, or between the base plate and the attachment or one of the attachments, and/or at least one of the valves extending through the base plate and/or through the attachment or one of the attachments. In the present application, an “attachment” is to be understood as a component other than the base plate, which is connected to the base plate by connecting elements, such as riveting, and preferably attaches the lines and/or the valves to the base plate or fixes them by clamping.

The provision of at least one attachment represents a simple and cost-effective way of forming the lines and valves as integral parts of the distributor unit by arranging them therein or between the base plate and the attachment or by extending them through the base plate and/or through the attachment. The lines and valves are furthermore better protected, which improves the robustness of the distributor unit. The provision of two attachments is structurally particularly advantageous.

The first and/or second filter unit preferably comprises filter cartridges each comprising a membrane filter, at least one inlet valve, at least one outlet valve and optionally at least one drain hose, wherein at least one of the respective inlet valves communicates with a corresponding connection of the distributor unit via a corresponding hose and/or at least one of the respective outlet valves is configured to communicate with the external radiation-shielded container for waste solution via the drain hose. The communication or connection preferably occurs by means of a one-piece hose connection, but multi-piece connections with intermediate additional components are also included.

The claimed structure of the filter units with inlet valves, outlet valves and membrane filters makes it possible to let liquid in and out in a controlled manner in order to be able to separate the filter units from the cartridge device after filtration without liquid accidentally escaping to the outside, which would pose a health risk. The membrane filter can then be removed and can be further treated and analysed separately to determine the microbial load. The provision of a drain hose furthermore has the advantage that liquid can be drained from the filter units into a shielded container for waste solution without contact with the ambient air.

The inlet valves and/or the outlet valves preferably comprise sterile filters to enable sterile aeration and/or deaeration of the respective filter units or filter cartridges.

The valves of the filter units can communicate with the ambient air via the sterile filters so that sterile aeration or deaeration of the filter units can take place, making it easier to fill the filter units with liquid or drain liquid therefrom. Due to the sterile filters, no contamination can enter the filter units from the outside, which would falsify the results.

The distributor unit of the cartridge device is preferably configured to be received by a first external actuator module, and the valves are preferably configured to be actuated in an automated manner by the first external actuator module or its sub-components, and/or the inlet valves and/or the outlet valves of the first and/or second filter unit are preferably configured to be received and actuated in an automated manner by a second external actuator module, and/or the syringe of the cartridge device is preferably configured to be received and actuated in an automated manner by an external syringe pump module or an actuating unit, and/or the drain hose of the cartridge device is preferably configured to be received by an external hose pump module. In the present application, a “module” is preferably to be understood as an apparatus unit that is assigned a specific task in an overall system, e.g. for sterility testing by means of membrane filtration. A plurality of modules can then be combined and assembled to form a complete system, e.g. for sterility testing by means of membrane filtration.

The compatibility of the distributor unit, the valves of the filter units, the syringe and the drain hose with corresponding external actuator or pump modules provides a simple and cost-effective way of automating membrane filtration for sterility testing.

All components of the cartridge device are preferably configured for single use or all components of the cartridge device, with the exception of the drain hose, are preferably configured for single use and the drain hose is configured or constructed as a permanent line and/or to be flushable.

Configuring the components for single use has the advantage that time and costs for cleaning after use can be saved and residues remaining after inadequate cleaning, which would falsify the test result, can be prevented. Since no liquid can pass from the drain hose into the rest of the cartridge device, it can also be reused without prior cleaning, thus saving costs.

A system according to the invention for the automated sterility testing of radiopharmaceuticals by means of membrane filtration comprises a cartridge device according to the invention, a container for/with buffer solution, a first container for/with nutrient solution and preferably a second container for/with nutrient solution, a radiation-shielded container for waste solution and a radiation-shielded container for sample material, wherein the corresponding hoses of the cartridge device communicate with the container for/with buffer solution, the first container for/with nutrient solution, the second container for/with nutrient solution, the radiation-shielded container for waste solution and the radiation-shielded container for sample material, preferably in order to transport liquids out of the containers or into the containers via the distributor unit of the cartridge device, optionally in an automated manner.

The provision of containers with buffer solution and nutrient solution as well as radiation-shielded containers for sample material and waste solution, which communicate with the corresponding hoses of the cartridge device, enables in particular the automated transport of liquids for the sterility testing of radiopharmaceuticals by means of membrane filtration. The radiation-shielded containers have the advantage that they shield the radioactive substances contained therein such that substantially no radioactivity can escape to the outside, thus improving personal protection. In particular radiation-shielded containers according to EP 3944867 A1 can be used, in particular as and for the waste container. To further improve radiation protection, the system can furthermore be housed in an isolator, preferably in accordance with EP 3944867 A1.

An arrangement according to the invention comprises a cartridge device according to the invention and a packaging in which the cartridge device is sterilely enclosed, the cartridge device being in a disassembled, partially disassembled or assembled state in the packaging.

The sterile packaging of the cartridge device ensures that the cartridge device is protected from external contamination that would falsify the results of the sterility testing. Providing the cartridge device in a disassembled or partially disassembled state may be advantageous for cost reasons, but it is preferred for it to be provided in an assembled state in order to prevent possible contamination due to manual assembly and to provide a “plug-and-play” solution.

A first use according to the invention relates to the use of the cartridge device or system according to the invention for the automated sterility testing of radiopharmaceuticals by means of membrane filtration.

A second use according to the invention relates to the use of the cartridge device or system according to the invention in an apparatus for the automated sterility testing of radiopharmaceuticals by means of membrane filtration.

The cartridge device according to the invention is particularly suitable for use for the automated sterility testing of radiopharmaceuticals by means of membrane filtration, in particular in a corresponding apparatus.

### BRIEF DESCRIPTION OF THE DRAWINGS

Further features and advantages of the present invention are apparent from the following description of the embodiments with reference to the accompanying figures. These figures show the following:

FIG. 1 an embodiment of a distributor unit of a cartridge device according to the invention, wherein possible connections and interconnections of the components of the distributor unit according to a preferred embodiment are also shown in a technically clear and relevant manner;

FIG. 2 an embodiment of a cartridge device according to the invention, wherein possible connections and interconnections of the components of the cartridge device according to a preferred embodiment are also shown in a technically clear and relevant manner;

FIG. 3 an implementation of an embodiment of the cartridge device according to the invention in an apparatus for the automated sterility testing of radiopharmaceuticals.

### DESCRIPTION OF PREFERRED EMBODIMENTS

The reference numbers used below as well as in the drawings and the patent claims are substantially to be regarded as informative and not limiting, in particular as regards the amount thereof. Irrespective thereof, at least partially identical letter components optionally indicate functional groups of the cartridge device according to the invention. For example, the reference numbers 3a, 4a and 5a indicate that the corresponding valve 3a, the corresponding connection 4a and the corresponding hose 5a form a functional sub-unit of the cartridge device and can communicate with one another. Similarly, reference number 5ab designates a line or line section between valves 3a and 3b.

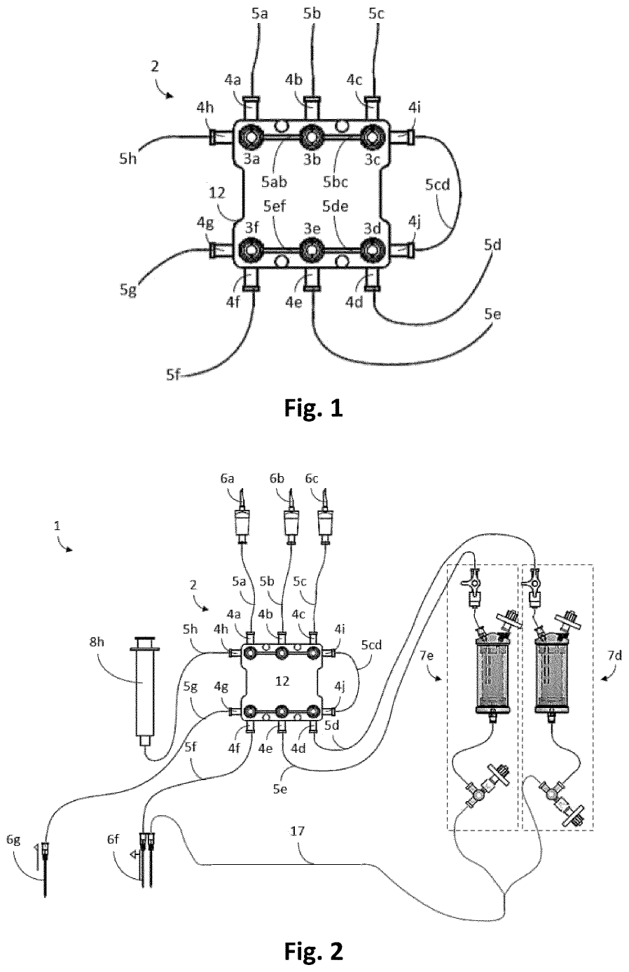

FIG. 1 shows an embodiment of a distributor unit (2) of a cartridge device (1) according to the invention, which can be connected to an actuator module (101) of an apparatus (100) for the automated production and/or sterility testing of radioactive substances, in particular radiopharmaceuticals.

The distributor unit (2) comprises a plurality of valves (3a-3f), in particular three-way valves, a plurality of lines (5ab-5ef) and a plurality of connections (4a-4i) which can selectively communicate with one another via the valves (3a-3f). The valves (3a-3f) are arranged on the distributor unit (2) in such a manner that when the cartridge device (1) is connected to an actuator module (101), they can be actuated by the actuators thereof in an automated manner.

In the embodiment shown by way of example in FIG. 1, the substantially rectangular distributor unit (2) comprises six three-way valves (3a-3f), eight or ten connections (4a-4i) and five internal or external lines (5ab-5ef) which connect the valves (3a-3f) to one another, four valves (3a, 3c, 3d, 3f) each being arranged in a corner and two valves (3b, 3e) each being arranged at an edge of the distributor unit (2). The configuration of the distributor unit (2), in particular the arrangement of the valves (3a-3f), is compatible with an actuator module of the type “SLM2” of the firm “Eckert & Ziegler”.

While the valves (3a, 3f) shown on the left in FIG. 1 are each connected to two connections and one line, the other valves (3b, 3c, 3d, 3e) are each connected to one connection and two lines. The connections (4i, 4j) shown on the right in FIG. 1 are optional since they can also be connected to one another via an internal line (5cd) instead of via an external line (5cd).

FIG. 2 shows an embodiment of a cartridge device (1) according to the invention which can be connected to an actuator module (101) of an apparatus (100) for the automated production and/or sterility testing of radioactive substances, in particular radiopharmaceuticals.

Five connections (4a, 4b, 4c, 4f, 4g) of the distributor unit (2) are connected to corresponding cannulas (6a, 6b, 6c, 6f, 6g) via corresponding hoses (5a, 5b, 5c, 5f, 5g), two connections (4d, 4e) of the distributor unit (2) are connected to corresponding filter units (7d, 7e) via corresponding hoses (5d, 5e), and one connection (4h) of the distributor unit (2) is connected to a syringe (8h) via a corresponding hose (5h).

The cannulas (6a, 6b, 6c, 6f, 6g) are configured to be connectable to a container for buffer solution (16a), a first container for nutrient solution (16b), a second container for nutrient solution (16c), a radiation-shielded container for waste solution (16f) and a radiation-shielded container for sample material (16g), respectively. The nutrient solutions in the first and second containers (16b, 16c) may be the same or different.

The filter units (7d, 7e) are detachably connected to the cartridge device (1) and each contain a filter cartridge with a membrane filter (8d, 8e), at least one inlet valve (9d, 9e) and at least one outlet valve (10d, 10e). In the shown embodiment, each of the filter units (7d, 7e) comprises two inlet valves (9d, 9e) in the form of two-way valves and one outlet valve (10d, 10e) in the form of a three-way valve.

While one of the respective two inlet valves (9d, 9e) is configured to open or close a fluid connection between the distributor unit (2) and the respective filter cartridge, the other of the respective two inlet valves (9d, 9e) is equipped with a sterile filter and is configured to open or close a fluid connection between the ambient air and the respective filter cartridge in order to facilitate the entry of liquid into and the drainage of liquid from the respective filter cartridge. Alternatively, only one inlet valve (9d, 9e) in the form of a three-way valve may be provided in each case, which selectively connects the inlet of the filter cartridge to the distributor unit (2) or the ambient air. The respective outlet valves (10d, 10e) are also equipped with sterile filters and are configured to selectively connect the drain hose (17) to the filter cartridges or the ambient air in order to facilitate the entry of liquid into and the drainage of liquid from the respective filter cartridge.

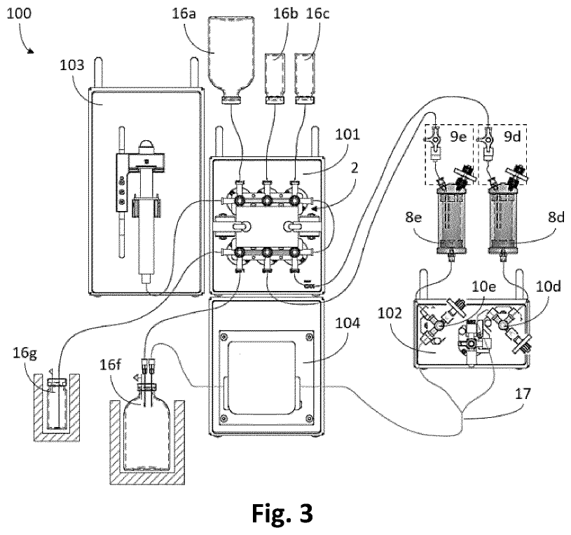

FIG. 3 shows an embodiment of a system according to the invention in an apparatus (100) for the automated sterility testing of radioactive substances, in particular radiopharmaceuticals. In addition to the cartridge device, the system according to the invention comprises a container for buffer solution (16a), a first container for nutrient solution (16b), a second container for nutrient solution (16c), a radiation-shielded container for waste solution (16f) and a radiation-shielded container for sample material (16g). The apparatus (100) comprises a first actuator module (101), a second actuator module (102), a first pump module (103) and a second pump module (104).

The distributor unit (2) of the cartridge device (1) is inserted into the first actuator module (101) of the apparatus (100) such that the valves (3a-3f) of the cartridge device (1) can be actuated in an automated manner by corresponding actuators of the first actuator module (101).

According to the nomenclature of FIGS. 1 and 2, the first connection (4a) is connected via the first hose (5a) and the first cannula (6a) to the container for buffer solution (16a), the second connection (4b) is connected via the second hose (5b) and the second cannula (6b) to the first container for nutrient solution (16b), the third connection (4c) is connected via the third hose (5c) and the third cannula (6c) to the second container for nutrient solution (16c), the sixth connection (4f) is connected via the sixth hose (5f) and the fourth cannula (6f) to the radiation-shielded container for waste solution (16f), and the seventh connection (4g) is connected via the seventh hose (5g) and the fifth cannula (6g) to the radiation-shielded container for sample material (16g).

The filter units (7d, 7e) of the cartridge device (1) are inserted into the second actuator module (102) of the apparatus (100) such that the outlet valves (10d, 10e) of the filter units (7d, 7e) can be actuated in an automated manner by corresponding actuators of the second actuator module (102).

The outlet valves (10d, 10e) are connected to the radiation-shielded container for waste solution (16f) via a common drain hose (17) or separate drain hoses. The drain hose (17) or drain hoses may be part of the cartridge device (1) and may be configured to communicate with the radiation-shielded container for waste solution (16f).

The syringe (8h) of the cartridge device (1) is inserted into the first pump module (103) of the apparatus (100) such that the syringe (8h) can be actuated in an automated manner by a corresponding actuator of the first pump module (103), by means of which liquid can be drawn from the system or delivered to the system. The first pump module (103) may therefore be a syringe pump module.

The drain hose (17) is inserted into the second pump module (104) of the apparatus (100) such that a corresponding actuator (not shown) of the second pump module (104) can interact in an automated manner with the drain hose (17) in order to generate a fluid flow therein, in particular in order to pump liquid out of the filter units (7d, 7e).

The cartridge device (1) is configured for single use, but could also be reused after thorough decontamination. The filter units (7d, 7e) are detachably connected to the cartridge device (1) so that they can be separated for further treatment and examination after filtration. By means of the inlet and outlet valves (9d, 9e, 10d, 10e), it can be ensured that no contaminated or radioactive liquid can escape from the filter units (7d, 7e).

In the context of a specific embodiment, the following components are particularly suitable for the cartridge device (1) according to the invention and can be expediently designated as follows for the person skilled in the art:
Base plate (2): “C2-Manifold”,
Hose (5a): “PE (polyethylene) extension, M/M (male/male) LL, 50 cm”,
Hoses (5b, 5c): “PE extension, M/F (male/female) LL, 20 cm”,
Hoses (5d, 5e): “PE extension, M/F LL, 30 cm”,
Hoses (5f, 5g): “PE extension, M/F LL, 20 cm”,
Hose (5h): “PE extension, M/F LL, 15 cm”,
Line (5cd): “PE extension, M/M LL, 10 cm”,
Cannula (6a): “Codan Microspike”,
Cannulas (6b, 6c): “Spikes Venting”,
Cannulas (6f, 6g): “Needles Sterican 0.9×70 mm 20 Gx1 ½”
Syringe (8h): “Syringe BD 10 ml”,
Sterile filter: “Hydrophobic Filter 0.2 μm PTFE 28213”,

Connection between filter cartridges and outlet valves: 2 “Luer connectors male PP, 1/16″ 1.6 mm” and “silicone hose 1×3 mm”.

Standardised membrane filtration using the cartridge device or system according to the invention can proceed and be carried out as follows. The valves of the distributor unit (2) are set such that there is a fluid connection between the syringe (8h) and the container with buffer solution (16a), and buffer solution is drawn from the corresponding container (16a) by actuating the syringe (8h). The valves of the distributor unit (2) are then set such that there is a fluid connection between the syringe (8h) and the container with sample material (16g), and the buffer solution is fed into the corresponding container (16g) by actuating the syringe (8h), where the sample material mixes with the buffer solution or dissolves therein.

Next, the buffer solution with the sample material dissolved therein is drawn back again, the valves of the distributor unit (2) are set such that there is a fluid connection between the syringe (8h) and the first filter unit (7d), and part of the buffer solution is conveyed into the first filter unit (7d). The valves of the distributor unit (2) are then set such that there is a fluid connection between the syringe (8h) and the second filter unit (7e), and the remainder of the buffer solution is conveyed into the second filter unit (7e). The filtrate from both filter units (7d, 7e) is fed into the container for waste solution (16f) through the drain hose (17).

The valves of the distributor unit (2) are then set such that there is a fluid connection between the syringe (8h) and the first container with nutrient solution (16b) and the first nutrient solution is drawn in. The valves of the distributor unit (2) are then set such that there is a fluid connection between the syringe (8h) and the first filter unit (7d), and the nutrient solution is conveyed into the first filter unit (7d). A corresponding procedure is repeated for the second nutrient solution (16c) and the second filter unit (7e).

The inlet valves (9d, 9e) of the filter units (7d, 7e) are then set such that the filter cartridges are separated from the rest of the system and connected to the ambient air via sterile filters. The remaining liquid from the filter units (7d, 7e) is conveyed through the drain hose (17) into the container for waste solution (16f) before the outlet valves (10d, 10e) are closed, the filter units (7d, 7e) are separated and the membrane filters are removed for further treatment and examination.

### LIST OF REFERENCE NUMBERS

1 Cartridge device
2 Distributor unit
3a-3f Valves, optionally three-way valves,
4a-4j Connections
5ab-5ef Lines
5a-5h Hoses
6a-6c, 6f-6g Cannulas
7d, 7e Filter units
8d, 8e Membrane filter in filter cartridge
9d, 9e Inlet valves
10d, 10e Outlet valves
12 Base plate
16a Container for buffer solution
16b First container for nutrient solution
16c Second container for nutrient solution
16f Container for waste solution
16g Container for sample material
17 Drain hose
100 Apparatus
101 First actuator module
102 Second actuator module
103 First pump module
104 Second pump module

1. A cartridge device for use in an apparatus for the automated sterility testing of radiopharmaceuticals by means of membrane filtration, comprising:
a distributor unit with a base plate, valves, in particular three-way valves, lines and connections, wherein the connections can selectively communicate with one another via the valves and lines,
a first filter unit, and
at least five hoses, that communicate with corresponding connections of the distributor unit,
wherein one hose communicates with the first filter unit and four hoses are each respectively configured communicate with a first external container with buffer solution, an external container with nutrient solution, an external radiation-shielded container for waste solution or an external radiation-shielded container for sample material.

2. The cartridge device according to claim 1, further comprising:
a second filter unit; and
a further hose communicating with a corresponding connection of the distributor unit and the second filter unit.

3. The cartridge device according to claim 1, further comprising:
a syringe for a syringe pump, and
a further hose communicating with a corresponding connection of the distributor unit and the syringe.

4. The cartridge device according to claim 1, further comprising:
a further hose communicating with a corresponding connection of the distributor unit and configured to be able to communicate with a second external container containing nutrient solution.

5. The cartridge device according to claim 1, further comprising:
at least four or at least five cannulas attached to the distal end of the corresponding hoses, wherein the cannulas are configured and provided to be able to communicate with the external container with buffer solution, the first external container with nutrient solution, the second external container with nutrient solution, the external radiation-shielded container for waste solution and the external radiation-shielded container for sample material.

6. The cartridge device according to claim 1, wherein the distributor unit comprises at least six valves, all in the form of three-way valves, and at least eight connections, optionally ten connections.

7. The cartridge device according to claim 6, wherein the distributor unit comprises:
a first, second, third, fourth and fifth line,
a first three-way valve configured to establish selective communication between a connection for supplying buffer solution, a connection for connection to the syringe and the first line,
a second three-way valve configured to establish selective communication between a connection for supplying a first nutrient solution, the first line and the second line,
a third three-way valve configured to establish selective communication between a connection for supplying a second nutrient solution, the second line and the third line,
a fourth three-way valve configured to establish selective communication between a connection for connection to the first filter unit, the third line and the fourth line,
a fifth three-way valve configured to establish selective communication between a connection for connection to the second filter unit, the fourth line and the fifth line, and
a sixth three-way valve configured to establish selective communication between a connection for the removal of waste solution, a connection for supplying sample solution and the fifth line.

8. The cartridge device according to claim 7, wherein:
at least one of the first, second, fourth and fifth lines, is an integral part of the distributor unit and the third line is an external hose or an integral part of the distributor unit, or
at least one of the first, second, third, fourth, fifth and sixth three-way valves is an integral part of the distributor unit.

9. The cartridge device according to claim 8, wherein the distributor unit furthermore comprises at least one attachment, at least two attachments, at least one of the lines being arranged inside the attachment or one of the attachments, or between the base plate and the attachment or one of the attachments, optionally fixed thereby, or at least one of the valves extending through the base plate or through the attachment or one of the attachments.

10. The cartridge device according to claim 1,
wherein the first or the second filter unit comprises filter cartridges each comprising a membrane filter, at least one inlet valve, at least one outlet valve and at least one drain hose,
wherein at least one of the respective inlet valves, communicates with a corresponding connection of the distributor unit via a corresponding hose or at least one of the respective outlet valves is configured to communicate with the external radiation-shielded container for waste solution via the drain hose.

11. The cartridge device according to claim 10, wherein the inlet valves or the outlet valves comprise sterile filters to enable sterile aeration or deaeration of the respective filter units.

12. The cartridge device according to claim 1,
wherein the distributor unit of the cartridge device is configured to be received by a first external actuator module, and the valves are configured to be actuated in an automated manner by the first external actuator module, or
wherein the inlet valves and/or the outlet valves of the first or second filter unit, are configured to be received and actuated in an automated manner by a second external actuator module, or
wherein the syringe of the cartridge device is configured to be received and actuated in an automated manner by an external syringe pump module or an actuating unit, or
wherein the drain hose of the cartridge device is configured to be received by an external hose pump module.

13. The cartridge device according to claim 1,
wherein all components of the cartridge device are configured for single use, or
wherein all components of the cartridge device, with the exception of the drain hose, are configured for single use and the drain hose is configured as a permanent line or to be flushable.

14. A system for the automated sterility testing of radiopharmaceuticals by means of membrane filtration, comprising:
a cartridge device according to one of the preceding claims,
a container with buffer solution,
a first container with nutrient solution and a second container with nutrient solution,
a radiation-shielded container for waste solution, and
a radiation-shielded container for sample material,
wherein the corresponding hoses of the cartridge device communicate with the container with buffer solution the first container with nutrient solution, the second container with nutrient solution, the radiation-shielded container for waste solution and the radiation-shielded container for sample material,
in order to transport liquids out of the containers or into the containers via the distributor unit of the cartridge device, optionally in an automated manner.

15. The cartridge device according to claim 1, further comprising:
a packaging in which the cartridge device is sterilely enclosed, the cartridge device being in a disassembled, partially disassembled or assembled state in the packaging.

16. A method of performing sterility testing of radiopharmaceuticals, the method comprising using the cartridge device according to claim 1 for automated sterility testing of radiopharmaceuticals by means of membrane filtration.

17. (canceled)
